(19) United States
(12) Patent Application Publication
(10) Pub. No.: US 2006/0172020 A1
(43) Pub. Date: Aug. 3, 2006

(54) Dietary supplement for promoting control of blood-sugar levels and associated pathology in type 2 diabeties

(76) Inventor: Arthur H.K. Djang, Jamestown, NY (US)

Correspondence Address: Arthur H .K. Djang;Sante International, Inc, 111 West Second Street, Suite 4000, Jamestown, NY, 14701, US

(21) Appl. No.: 10/563,713

(22) PCT Filed: Jul. 13, 2004
(86) PCT No.: PCT/US04/22330

§ 371 Date: Jan. 7, 2006

Related U.S. Application Data
(60) Provisional application No. 60/488,492, filed on Jul. 17, 2003.

(51) Int. Cl. A61K 36/82; A61K 36/42; A61K 36/605

(52) U.S. Cl. 424/725; 424/729; 424/758; 424/777

(57) ABSTRACT

Provided is an herbal extract-based composition comprising an extract of Gynostemma pentaphyllum, an extract of Crataegus pinnatifidia (hawthorn), an extract of Camellia sinensis (green tea), and an extract of Momordica charantia (bitter melon). The composition may further comprise an extract of mulberry (Morus species). Also provided is a process for preparing a herbal extract-based composition which comprises separately extracting each of hawthorn, green tea, Gynostemma pentaphyllum, mulberry, and bitter melon; drying extraction eluates obtained from the extracting of each of the herbal components to obtain organic residues in forming a hawthorn extract powder, green tea extract powder, a Gynostemma pentaphyllum extract powder, a mulberry extract powder, and a bitter melon powder; and combining the green tea extract powder, the Gynostemma Pentaphyllum extract powder, the hawthorn extract powder, the mulberry extract powder, and the bitter melon powder in desired proportions to form the herbal extract-based composition which, when taken orally, has health-promoting effects including anti-diabetic effects that include, but are not limited to, decreasing visceral fat, reducing hyperglycemia, and reducing the occurrence and severity of diabetic complications, associated with type 2 diabetes.

### FIELD OF THE INVENTION

The present invention relates to an herbal extract-based composition comprised of a combination of extracts that may include an extract of Gynostemma pentaphyllum, an extract of Crataegus pinnatifidia, an extract of Camellia sinensis, an extract of mulberry (Morus species), and an extract of bitter melon (Momordica charantia). More particularly, the present invention provides a composition comprised of green tea polyphenols, hawthorn polyphenols, saponins of Gynostemma pentaphyllum, and an extract of bitter melon; and may further comprise N-containing sugars of mulberry. The present invention also provides a method of making and using the composition for promoting health.

### BACKGROUND OF THE INVENTION

Generally, herbal supplements are natural, safe when taken as recommended, and less expensive and sometimes more effective alternatives to drugs. These plant-based pharmaceuticals are used for medicinal purposes; and/or dietary supplements for disease prevention, for relief of ailments, for correction of metabolic abnormalities, and for health maintenance (collectively “health-promoting”). Gynostemma pentaphyllum, Crataegus pinnatifidia, Camellia sinensis, mulberry, and bitter melon each have been used individually for particular therapeutic applications.

1. Gynostemma pentaphyllum

Gynostemma pentaphyllum, also known as 5-leaf ginseng or jiaogulan or southern ginseng, is from the cucumber family and has traditionally been grown in a mountainous region in South Central China. This herb, a completely different plant than ginseng, is rich in special saponins termed “gypenosides” which are similar, and some identical, to the ginsenosides found in ginseng, but at a level several fold higher. These saponins have been shown to have antioxidant/cell protective effects. More particularly, the saponins protected cell membranes and cytosols, from oxidative injury, neutralize free radicals, helped preserve immune function during irradiation, lowered blood pressure, reduced vascular resistance, effects anti-platelet-aggregation, and reduced levels of serum triglycerides and total cholesterol.

2. Crataegus pinnatifidia

The leaves and berries of Crataegus pinnatifidia, also known as hawthorn, have been used traditionally for the treatment of heart conditions and for cardiovascular health. The hawthorn fruits (berries), known as “Shan-zha, have been used to improve digestion, and to alleviate various stomach ailments. Saponins, flavonoids (including hyperoside), and anthocyanins (including proanthocyanidins) extracted form hawthorn fruits have also shown cardiotonic (heart stimulating and regulating) activity including inhibition of arrhythmia, normalization of blood pressure, dilation of blood vessels and increase in coronary blood flow, reduction of serum triglyceride and cholesterol levels, reduction in symptoms of angina, and improvement of circulation.

3. Camellia sinensis

Dried leaves from the Camellia sinensis plant is processed into three types of tea: oolong tea, black tea, and green tea. In making green tea, the tea leaves are stabilized by moist or dry heat which destroys the enzyme polyphenoloxidase and thus, prevents oxidation of polyphenols. These polyphenols are the main biologically active ingredients in green tea, and include, but are not limited to catechins and/or epicatechins. Catechins, a chemical group of polyphenols possessing antioxidant properties (protects cells from free radical-mediated damage), include epigallocatechin-3 gallate (EGCG), epigallocatechin, and epicatechin-3-gallate. Recently, EGCG has been shown to be an inhibitor of urokinase, and enzyme crucial for cancer growth. The polyphenols in green tea, accounting for as much as 40% of tea's dry solids, have also been shown to reduce serum cholesterol and LDL (low density lipoprotein). Green tea polyphenols have been shown to prevent microbial (bacterial and viral) infections. For example, green tea polyphenols damage bacterial membranes. Further, extracts of green tea have been shown to prevent cancers of the lung, breast, prostate, liver, skin, esophagus, and colon. Green tea is also high in cavity-fighting fluoride- the amount of tea used to prepare one cup has approximately 0.3 milligrams of fluoride.

4. Mulberry

Mulberry leaves and berries have been found to contain flavonoids, identified as morusin, kuwanon H, morusin-4′-glucoside, which have demonstrated anti-HIV activity. Also another chemical isolated from mulberry, deoxynojirimycin (“DNJ”), inactivates HIV. DNJ is a potent alpha glucosidase inhibitor which inhibits glucosidase I. DNJ is currently being evaluated as a supplement for use in preventing diabetes. Galactolipid compounds and a triterpene compound, contained in methanol extracts of mulberry leaves, have hypoglycemic activity when tested in streptozotocin-induced diabetic rats. Mulberry also contains resveratrol, a powerful antioxidant known to have anti-cancer effects (e.g., against skin cancer and leukemias), and to lower risk of heart attacks and heart disease.

5. Bitter Melon

Bitter melon (Momordica charantia) is fruit indigenous to South America and Asia. Bitter melon appears to contain components having structural similarity to insulin, and has been used in alternative therapy for lowering glucose levels in individuals with diabetes mellitus. Hypoglycemic effects have been noted with ingestion of powdered bitter melon fruit or bitter melon juice or extracts of bitter melon leaves. One of bitter melon's components, charantin, is composed of mixed steroids that reportedly are more potent in action than the antidiabetic drug tolbutamid. Bitter melon is a component of the regular diet of the native population of Okinawa, a population having one of the highest life expectancies in the world.

A herbal extract-based composition comprising a novel combination of Gynostemma pentaphyllum, Crataegus pinnatifidia, Camellia sinensis has been described previously (U.S. Pat. Nos. 5,910,308, 6,168,795, and 6,713,094 to the present inventor and assignee); and Gynostemma pentaphyllum, Crataegus pinnatifidia, Camellia sinensis, mulberry, and bitter melon have been used individually for health promoting and therapeutic purposes. However, not described is an herbal extract-based composition comprised of a combination of an extract of Gynostemma pentaphyllum, an extract of Crataegus pinnatifidia (hawthorn berries and/or leaves), an extract of Camellia sinensis (green tea), and an extract of bitter melon (Momordica charantia; one or more of fruit, seed, or leaves), and may further include an extract of mulberry (Morus species; berries and/or leaves), for health-promoting and/or therapeutic uses.

### SUMMARY OF THE INVENTION

This invention relates to a herbal extract-based composition that comprises a combination of components (in an amount expressed in percent by weight of the composition) wherein a first component comprises about 5 percent to about 20 percent by weight of Gynostemma pentaphyllum extract; a second component comprises about 20 percent to about 40 percent by weight of green tea extract; a third component comprises about 20 percent to about 40 percent by weight of hawthorn (berries and/or leaves) extract; a fourth component comprises about 20 percent to about 40 percent by weight of bitter melon (fruit and/or seeds and/or leaves) extract; and optionally, a fifth component comprises about 0 percent to about 15 percent by weight of mulberry (berries and/or leaves) extract. Preferably, the composition comprises about 5 percent by weight of Gynostemma pentaphyllum extract, about 30 percent by weight of green tea extract, about 30 percent by weight of hawthorn extract, about 30 percent by weight of bitter melon extract, and about 0 to 10 percent by weight of mulberry extract.

In another embodiment of the present invention, provided is a composition comprising, as active components, green tea polyphenols, hawthorn polyphenols, saponins of Gynostemma pentaphyllum, and an extract of bitter melon; and may further comprise N-containing sugars of mulberry. The composition preferably comprises about 5 percent to about 20 percent by weight of Gynostemma pentaphyllum saponins, about 20 percent to about 40 percent by weight of green tea polyphenols; about 20 percent to about 40 percent by weight of hawthorn polyphenols; about 20 percent to about 40 percent by weight of bitter melon (fruit and/or seeds and/or leaves) extract; and about 0 percent to about 15 percent by weight of mulberry N-containing sugars. More preferably, the composition according to the present invention comprises about 5 percent by weight of Gynostemma pentaphyllum saponins; about 30 percent by weight of green tea polyphenols; about 30 percent by weight of hawthorn polyphenols; about 30 percent by weight of bitter melon (fruit and/or seeds and/or leaves) extract; and about 0 percent to about 10 percent by weight of mulberry N-containing sugars.

Another aspect of the present invention is a process for preparing the herbal extract-based composition. Essentially, this method comprises separately extracting each herbal component (hawthorn berries and/or leaves, green tea leaves, leaves of Gynostemma pentaphyllum, mulberry berries and/or leaves, and fruit and/or seeds and/or leaves of bitter melon); drying the extraction eluates to obtain the organic residues in forming a hawthorn extract powder, green tea extract powder, a Gynostemma pentaphyllum extract powder, a mulberry extract powder and a bitter melon extract powder; and combining the green tea extract powder, the Gynostemma pentaphyllum extract powder, the hawthorn extract powder, the mulberry extract powder, and the bitter melon extract powder in desired proportions to form the herbal extract-based composition. In one embodiment, this method comprises the steps of:
(a) separately extracting a first batch of macerated (e.g., cut or tom) hawthorn (berries and/or leaves), macerated green tea leaves, macerated Gynostemma pentaphyllum leaves, macerated mulberry (berries and/or leaves), and macerated bitter melon (fruit and/or leaves and/or seed) in warm (greater than room temperature) water;
(b) recovering a first extract eluate from the respective extraction of each herbal component;
(c) repeating step (a), recovering a second extract eluate for each respective herbal component, and pooling the first and second extract eluates of the respective herbal component;
(d) separately extract a second batch of each of macerated hawthorn, macerated green tea leaves, macerated Gynostemma pentaphyllum leaves, macerated mulberry, and macerated bitter melon in an aqueous alcohol (e.g., 70% ethanol);
(e) recovering a first aqueous alcohol extract eluate from the respective aqueous alcohol extraction of each herbal component;
(f) repeating step (d), recovering a second aqueous alcohol extract eluate from the respective aqueous alcohol extraction of each herbal component, and pooling the second aqueous alcohol extract eluate with the respective first aqueous alcohol extract eluate from each herbal component;
(g) recovering the organic residue of each herbal component by reducing the liquid portion of each of the respective pooled eluates by drying (air drying, freeze drying or a combination thereof), in forming a green tea extract powder, a Gynostemma pentaphyllum extract powder, a hawthorn extract powder, a mulberry extract powder, and a bitter melon powder;
(h) combining the green tea extract powder, the Gynostemma pentaphyllum extract powder, the hawthorn extract powder, the mulberry extract powder, and the bitter melon powder in the desired proportions. It is noted that the pooled water extract eluate and the pooled aqueous alcohol extract eluate from each herbal component were dried separately and then the organic residues for that herbal component combined to form the extract powder for that herbal component; or alternatively, the pooled water extract eluate and the pooled aqueous alcohol extract eluate from each herbal component may be combined and then dried to form the extract powder for that herbal component. In yet another alternative, any of one or more of the components may be made singly, or in combination with one or more of the other components, before combining with the remaining components in the desired proportions in forming the herbal extract-based composition. Alternately, for producing a bitter melon extract powder, the fruit of the bitter melon may be cut up into small pieces, which are then freeze-dried and powdered using standard methods known in the art. This technique involving freeze drying may also be used for producing a hawthorn extract powder from hawthorn berries, and a mulberry extract powder from the berries of mulberry.

Using such a method, produced is an extract of green tea comprising green tea polyphenols; produced is an extract of Gynostemma pentaphyllum comprising Gynostemma pentaphyllum saponins; produced is an extract of hawthorn comprising hawthorn polyphenols; produced is an extract of mulberry comprising mulberry N-containing sugars; and produced is an extract of bitter melon.

### DETAILED DESCRIPTION OF THE INVENTION

Definitions

In describing embodiments of the present invention, such terms as “first”, “second”, “third”, “fourth” and the like are words of convenience in order to distinguish between different elements. Such terms are not intended to be limiting as to the sequence of a method or priority in which the different elements may be utilized.

The term “saponins” is used herein, in relation to Gynostemma pentaphyllum and for purposes of the specification and claims, to mean triterpenoids containing a carbohydrate moiety, and steroids containing a carbohydrate moiety, as may be contained in Gynostemma pentaphyllum. As an example well known in the art, gypenosides are triterpenoid saponins contained in an extract of Gynostemma pentaphyllum.

The term “polyphenols” is used herein, in relation to Crataegus pinnatifidia (hawthorn) and for purposes of the specification and claims, to mean polyphenols which may include, but are not limited to, proanthocyanidins, chlorogenic acid, epicatechin, ursolic acid, rutin, quercetin, hyperoside, and isoquercitrin, as may be contained in hawthorn.

The term “N-containing sugars” is used herein, in relation to mulberry and for purposes of the specification and claims, to mean imino sugars and/or polyhydroxylated alkaloids isolated from mulberry. These may include, but are not limited to, one or more of nojirimycin, deoxynojirimycin, N-methyl-1-deoxynojirimycin, fagomine, 3-epi-fagomine, 1,4-dideoxy-1,4-imino-D-arabinitol, 1,4-dideoxy-1,4-imino-D-ribitol, calystegin C1, calystegin B2, 1,5dideoxy-1,5-imino-D-glucitol, and related alkaloid glycosides and glucosidase inhibitors.

The terms “hypoglycemic effect” and “anti-hyperglycemic effect” are used interchangeably, for purposes of the specification and claims, to mean the reduction of blood glucose levels, as a result of administration of the composition according to the present invention, in an individual with a hyperglycemic condition (e.g., an individual having type 2 diabetes, or a hyperglycemic condition induced in an experimental animal model). Examples of such reduction in blood glucose levels are illustrated in more detail herein.

The term “anti-diabetic effect” is used, for purposes of the specification and claims, to mean reduction or inhibition of one or more of the conditions or complications associated with type 2 diabetes (or an experimental animal model for type 2 diabetes or a pre-diabetic condition) as known to those skilled in the art, including but not limited to hyperglycemia, and/or an increase in visceral fat (adiposity).

The present invention relates to a novel herbal extract-based composition that comprises a combination of components (in an amount expressed in percent by weight of the composition) wherein a first component comprises about 5 percent to about 20 percent by weight of Gynostemma pentaphyllum extract; a second component comprises about 20 percent to about 40 percent by weight of green tea extract; a third component comprises about 20 percent to about 40 percent by weight of hawthorn extract; a fourth component comprises about 20 percent to about 40 percent by weight of bitter melon extract; and optionally, a fifth component comprises about 0 percent to about 15 percent by weight of mulberry extract. Preferably, the composition comprises about 5 percent by weight of Gynostemma pentaphyllum extract, about 30 percent by weight of green tea extract, about 30 percent by weight of hawthorn berries extract, about 30 percent by weight of bitter melon, and about 0 to 10 percent by weight of mulberry (leaves and/or berries) extract. More preferably, the composition of the present invention comprises, as active components, green tea polyphenols, hawthorn polyphenols, saponins of Gynostemma pentaphyllum, and an extract of bitter melon; and may further comprise N-containing sugars of mulberry. The composition preferably comprises, as active ingredients, about 5 percent to 20 percent by weight of saponins of Gynostemma pentaphyllum, about 20 percent to about 40 percent by weight of green tea polyphenols; about 20 percent to about 40 percent by weight of hawthorn polyphenols; about 20 percent to about 40 percent by weight of bitter melon extract; and about 0 percent to about 15 percent by weight of mulberry N-containing sugars. More preferably, the composition according to the present invention comprises about 5 percent by weight of Gynostemma pentaphyllum saponins; about 30 percent by weight of green tea polyphenols; about 30 percent by weight of hawthorn polyphenols; about 30 percent by weight of bitter melon extract; and about 0 percent to about 10 percent by weight of mulberry N-containing sugars. As apparent to those skilled in the art, depending on the form of the composition (e.g., liquid, capsule, lotion, etc.), the active ingredients may comprise from about 10% to about 100% of the composition, with any remaining portion being inactive ingredients (e.g., a base or carrier for delivering the active Ingredients) as will be more apparent from the following descriptions.

The composition of the present invention provides a novel combination of herbal components which work together effectively in producing an anti-diabetic effect. It is believed that this novel combination produces a surprisingly effective anti-diabetic effect through the interaction of its component parts in an unexpected, cooperative manner rather than as an additive manner. For example, the component comprising green tea polyphenols act in concert with the other components (active ingredients) of the composition according to the present invention, and contribute to improve insulin sensitivity, lower blood glucose concentrations in individuals with hyperglycemia, and contribute to loss of accumulated visceral fat, in producing an anti-diabetic effect. In particular, green tea polyphenols comprising catechins are believed to be insulin sensitizers (including, but not limited to, increasing the number of insulin receptors on cells expressing insulin receptors at their surface), help delay of glucose absorption, repress hepatic glucose production, and reduce visceral fat accumulation; all effects contributing to the anti-diabetic effect of the composition according to the present invention. The component comprising hawthorn polyphenols act in concert with the other components of the composition according to the present invention, and contributes to improve insulin sensitivity, and lower blood glucose concentrations in individuals with hyperglycemia (e.g., can significantly reduce fasting blood glucose levels when consumed by an individual with type 2 diabetes, as compared to the fasting blood glucose levels of the same individual whom did not consume the component), in producing an anti-diabetic effect. In particular, hawthorn polyphenols comprising chlorogenic acid are believed to lessen a hyperglycemic peak (i.e., lower blood glucose levels in hyperglycemia, the abnormally high blood glucose levels found, for example, in individuals with type 2 diabetes), reduce hepatic glycogenolysis (conversion by the liver of glycogen into glucose), and reduce glucose absorption; all effects contributing to the anti-diabetic effect of the composition according to the present invention. The component comprising saponins of Gynostemma pentaphyllum act in concert with the other components of the composition according to the present invention, and contributes to improve insulin sensitivity, and lower blood glucose concentrations in individuals with hyperglycemia, in producing an anti-diabetic effect. In particular, such saponins are believed to lower blood glucose levels in an individual having hyperglycemia, and reduce insulin-stimulated lipogenesis; all effects contributing to the anti-diabetic effect of the composition according to the present invention. The component comprising an extract of bitter melon acts in concert with the other components of the composition according to the present invention, and contributes to hypoglycemic effects in individuals with hyperglycemia, in producing an anti-diabetic effect. In particular, extract of bitter melon is believed to lower blood glucose levels in an individual consuming such extract; an effect contributing to the anti-diabetic effect of the composition according to the present invention. Optionally, and preferably, the composition of the present invention further comprises a mulberry extract as a component of the composition. The component comprising mulberry N-containing sugars acts in concert with the other components of the composition according to the present invention, and contributes to anti-hyperglycemic effects in individuals with hyperglycemia; an effect contributing to the anti-diabetic effect of the composition according to the present invention.

Thus, the herbal extract-based composition of the present invention is an arrangement and unique combination that has been found to provide a combined, unexpected therapeutic or health-promoting benefits, particularly when the herbal extract-based composition is taken by an individual as a dietary supplement. Depending on the desired use of the composition according to the present invention to provide one or more particular health-promoting and/or therapeutic effects, the composition can be provided as a component having the main therapeutic or health promoting activity (e.g., as active ingredients) contained in a form including, but not limited to, a tea, a liquid extract, a beverage, a gum, a lozenge, a powder, and a tablet (including capsule, caplet, or other pill type form), and a lotion. In one embodiment, the composition according to the present invention is included in a chewing gum formulation. The composition of chewing gum is conventional, and well known to those skilled in the art. For example, a gum base that may be mixed with the composition includes a base comprised of arabic, guar, natural rubber gums; sweeteners (e.g., sugar, stevia, saccharin, sorbitol, sugar substitute(s), aspartame); flavoring agents (e.g., mints, fruits), coloring agents; or a combination thereof. In another embodiment, the composition is used as a tea.

In another embodiment, the composition according to the present invention is included in a beverage formulation. The composition of beverages are conventional, and well known to those skilled in the art. For example, an aqueous carrier that may be mixed with the composition includes carriers comprised of spring water, filtered water, distilled water, carbonated water, flavored water, “sports” drinks (drinks containing carbohydrates and salts, as known in the art) juices, or combinations thereof. Additionally, the beverage may further comprise components known to the beverage industry including preservative agents, sweeteners, flavoring agents, coloring agents, and combinations thereof.

In another embodiment, the composition according to the present invention is in liquid extract form. The composition of liquid extracts are conventional, and well known to those skilled in the art. For example, an aqueous carrier that may be mixed with the composition includes carriers comprised of spring water, filtered water, or distilled water. Additionally, the liquid extract may further comprise components including preservative agents, sweeteners, flavoring agents, coloring agents, and combinations thereof.

In another embodiment, the composition according to the present invention may be incorporated into a tablet (including capsule, caplet, pill, and the like). Suitable bases are known to those skilled in the art to include fillers, binders, coatings, excipients and combinations thereof. For example, base ingredients may include, but are not limited to, plant cellulose, natural silica, magnesium stearate, wax, vegetable glycerides, vegetable stearate, and a combination thereof.

In another embodiment, where topical application to the skin or mucous membranes is desired to provide health-promoting effects, the composition may be incorporated into a cream or ointment base. Suitable bases are known to those skilled in the art to include one or more of purified water, lanolin, propylene glycol, mineral oil, tea oil, vegetable or flower oils, glycerin, glyceryl stearate, cetyl alcohol, propylparaben, preservatives, fragrance, and the like. Formulations containing the composition according to the present invention may comprise topical agents including, but not limited, to a rinse, a cream, an ointment, a gel, and a suppository.

It will be appreciated by those skilled in the art that the amount of the composition in the any of the forms herein described will depend on the type of form, other ingredients in the form, the mode of administration of the form, and the desired health-promoting or therapeutic effect to be provided. For example, typically, the composition according to the present invention will comprise, as an active ingredient, from about 10% to about 100% of the form (e.g. in weight percent or weight per volume).

The therapeutic and/or health-promoting benefits provided by the arrangement and unique combination comprising the composition according to the present invention may be more apparent by the following examples which are provided for purposes of illustration, and not limitation.

### EXAMPLE 1

Recent estimates indicate that during the last decade, the incidence of type II diabetes has increased in Americans by about 40%. Additionally, the incidence of the disease in adults in their 30s has increased by 70%. Major predisposing factors to developing type II diabetes include being overweight (current estimates indicate that 61% of Americans are overweight), insulin resistance, and diets high in refined carbohydrates. In diabetic individuals, increased blood concentration of glucose (hyperglycemia) is known to induce pathological complications, including heart disease (particularly as a result of constricting of blood vessels, and atherosclerosis), kidney disease (nephropathy), stroke, peripheral vascular disease, and eye and nerve complications (neuropathy). Recent studies indicate that strict glucose control can have long term benefits by helping diabetic individuals avoid the complications of diabetes. A combination of diet, exercise, and dietary supplements has been promoted as a means by which diabetics can control their blood sugar levels and manage their disease.

The herbal extract-based composition of the present invention contains a unique combination of components working together to provide health-promoting effects for individuals with diabetes. For example, the composition according to the present invention possess hypoglycemic activity, and more preferably may selectively lower blood glucose levels which are elevated. It is commonly accepted that normal fasting blood glucose levels in an individual range from about 70 to about 110 milligrams of glucose per deciliter (“dl”) of blood. Elevated (“hyperglycemic”) blood glucose levels occur in individuals which are classified as pre-diabetics (having a fasting blood glucose level in the range of 110 to about 125 mg/dl); and in individuals having diabetes (having a fasting blood glucose level greater than 125 mg/dl). Preferably, the composition according to the present invention lowers elevated blood glucose levels, while not significantly modifying normal blood glucose levels. Additionally, the herbal extract-based composition according to the present invention possesses a plurality of antioxidants such as saponins, flavonoids, and anthocyanins (including proanthocya-nidins). These antioxidants enhance the function of the liver, an organ that works with the pancreas to regulate glucose levels in an individual. Additionally, the antioxidants and other beneficial ingredients of the herbal extract-based help to prevent complications of diabetes by neutralizing free radicals. For example, these ingredients can dilate blood vessels, reduce atherosclerosis, promote the health of endothelium, and increase coronary blood flow in inhibiting the blood vessel constriction and heart complications induced by hyperglycemia, in promoting an anti-diabetic effect.

Also, the composition according to the present invention may be used as a dietary supplement for promoting an anti-hyperglycemia effect, in other conditions characterized by hyperglycemia. For example, it is now well established that most patients who have just completed major surgery have elevated blood glucose concentrations. It is believed that the hyperglycemia contributes to surgical complications such as an increased risk of infections. For example, in a study of surgical patients who stayed 6 days or longer in Intensive Care Units, deaths occurred at twice the rate when patients' blood sugar concentrations were twice normal values, as compared to the death rates of patients whose blood sugar concentrations were kept near normal concentrations. Thus, a dietary supplement that can help to control blood glucose levels in patients recovering from surgery may be desirable.

To illustrate the enhanced (e.g., unexpected efficiency of the) hypoglycemic activity of the composition according to the present invention, the herbal extract-based composition was in powder form. Individuals having hyperglycemia took a half a teaspoon of the powder as an oral, daily, dietary supplement in conjunction with a consistent diet (no significant dietary changes during the evaluation period). The average fasting blood glucose level was about 140 mg/dl in the individuals before taking the composition according to the present invention. After 2 days of taking the herbal extract-based composition according to the present invention, the average fasting blood glucose level in the individuals was normalized to 110 mg/dl, and continued to remain normalized during the evaluation period.

### EXAMPLE 2

In this embodiment, illustrated is the effect of the composition according to the present invention in alloxan-treated rats, a standard animal model for type 2 diabetes, as accepted by those skilled in the art. Tested was a composition comprising about 5 percent by weight of Gynostemma pentaphyllum saponins; about 35 percent by weight of green tea polyphenols, about 10 percent by weight of hawthorn polyphenols, about 30 percent by weight of bitter melon extract, and about 20 percent by weight of a mulberry extract. The herbal extract-based composition according to the present invention was used to produce an anti-diabetic effect; e.g., by efficaciously lowering blood glucose levels, as well as to effect other health-promoting responses, in treatment of hyperglycemia in an accepted model of type 2 diabetes. In this example, the composition was mixed in water and the alloxan-induced diabetic rats were orally administered the liquid containing the composition (at about 400 mg daily) as a dietary supplement via a gastric tube for every day of the study, as described herein in more detail. As a control for the test, rats not receiving the composition according to the present invention instead received water via a gastric tube. All rats were fed a normal diet of rat chow. The composition or the water was each administered to three different groups of rats. Group A received the herbal extract-based composition (6 rats) or the water (6 rats), and had blood glucose assessed before receiving alloxan. Group B received the herbal extract-based composition (6 rats) or the water (6 rats) seven days after receiving alloxan, and then blood glucose was assessed. Group C received the alloxan first, and then received either the herbal extract-based composition (6 rats) or the water (6 rats) 4 days later, and then blood glucose was assessed. Alloxan was administered intravenously at a single dose of 30 mg/kg.

The results of treatment, as measured by average fasting blood glucose levels (expressed in mg/dl±standard deviation), are Illustrated in Table 1.

TABLE 1

| Liquid received | Group A | Group B | Group C |
|---|---|---|---|
| water | 81.5 ± 7.5 | 190.3 ± 18.3 | 287.5 ± 20.6 |
| dietary supplement | 77.9 ± 7.3 | 181.3 ± 16.5 | 140.0 ± 12.7 |

It is apparent from these results, that the dietary supplement composition according to the present invention can produce an anti-diabetic effect by significantly reducing hyperglycemia in diabetics, as demonstrated with alloxan-induced diabetic rats. A similar anti-diabetic effect can be seen with the composition according to the present invention, but lacking a mulberry extract. It was also observed that on average there was a 10 percent decrease in the weight of alloxan-induced diabetic rats. However, treatment of alloxan-induced diabetic rats with the herbal extract-based composition according to the present invention resulted in only a slight decrease in the overall weight of the treated rats, and notably a decrease in visceral fat.

Toxicology studies were performed, in comparing rats receiving the composition according to the present invention (e.g., as a liquid via gastric tube at about 400 mg daily) (“Group A”) versus rats receiving water (“Group B”). Both groups received normal rat chow. After 90 days, rats receiving the composition according to the present invention showed no gross nor histopathological changes in the central nervous system, heart, lungs, kidneys, liver or spleen. Table 2 shows a comparison of the two groups in average body weight (“ABW”; in grams) at the initiation of the study (time 0), 30 days, 60 days, and 90 days; the average fasting blood glucose I(“ABG”; in mg/dl) level at 90 days; and the average blood cholesterol (“ABC”; in mmol/L) level at 90 days.

TABLE 2

| Group | ABW-0 | ABW-30 | ABW-60 | ABW-90 | ABG | ABC |
|---|---|---|---|---|---|---|
| A | 201 | 240 | 260 | 301 | 105 | 1.60 |
| B | 202 | 239 | 265 | 311 | 111 | 1.65 |

As shown in Table 2, there is only a slight decrease in average body weight between the rats receiving the composition according to the present invention and the rats which did not. Of note is that the reference value for fasting blood glucose levels of the rats is 111±6 mg/dl. Thus, it appears that the composition according to the present invention did not significantly change the fasting blood glucose levels in rats that were not diabetic (as shown in Table 2) as compared to the hypoglycemic effect noted in diabetic rats that received the composition according to the present invention (as shown in Table 1).

### EXAMPLE3

In this embodiment is illustrated a process for preparing the herbal based-extract of the present invention. In this process of preparation, selectively extracted from each component herb are compounds (some yet to be chemically defined) with particular types of pharmacological and/or health promoting activities. Using this process results in extracts that contain high concentrations of these compounds that comprise active ingredients in the herbal extract-based composition of the present invention. The extraction process of the present invention selectively extracts target compounds of desired pharmacological and/or health promoting activity, and thus, the extraction process should not be considered a “traditional extraction”; and the resultant extract is more appropriately viewed as a selective concentration of a combination of active components rather than a “total extract”. Thus, using the method according to the present invention is an approach to control the quality of, and standardize the composition of the target compounds to be selectively extracted for, the herbal extract-based composition of the present invention.

The method according to the present invention comprises: separately extracting each of hawthorn (berries and/or leaves), green tea (leaves), Gynostemma pentaphyllum (leaves), mulberry (berries and/or leaves) (if included in the composition), and bitter melon (leaves and/or seed and/or fruit); drying extraction eluates obtained from the extracting of each of hawthorn, green tea, Gynostemma pentaphyllum, mulberry, and bitter melon to obtain organic residues in forming a hawthorn extract powder, green tea extract powder, a Gynostemma pentaphyllum extract powder, a mulberry extract powder, and a bitter melon powder; and combining the green tea extract powder, the Gynostemma pentaphyllum extract powder, the hawthorn extract powder, the mulberry extract powder, and the bitter melon powder in desired proportions to form the herbal extract-based composition. In one embodiment of the present invention, the method comprises the steps of:
(a) separately extracting a first batch of macerated hawthorn, macerated green tea, macerated Gynostemma pentaphyllum, macerated mulberry, and macerated bitter melon (each referred to as a “herbal component”) in warm (greater than room temperature) water;
(b) recovering a first extraction eluate from the respective extraction of each herbal component;
(c) re-extracting each herbal component by repeating step (a), recovering a second extraction eluate, and pooling the second extraction eluate with the first extraction eluate of the respective herbal component;
(d) separately extracting a second batch of macerated hawthorn, macerated green tea, macerated Gynostemma pentaphyllum, macerated mulberry, and macerated bitter melon in an aqueous alcohol (e.g., 70% ethanol), and recovering alcohol extraction eluates for each herbal component;
(e) recovering the organic residue from each of the extraction eluates by reducing the liquid portion of each of the extraction eluates by drying (air drying, freeze drying, or a combination thereof) in forming a green tea extract powder, a Gynostemma pentaphyllum extract powder, a hawthorn extract powder, and a mulberry extract powder and a bitter melon extract powder;
(f) combining the green tea extract powder, the Gynostemma pentaphyllum extract powder, the hawthorn extract powder, the mulberry extract powder, and the bitter melon powders in the desired proportions to produce the herbal extract-based composition according to the present invention.
   In one embodiment, the extraction eluate produced for a herbal component using a water extraction may first be combined with the extraction eluate produced for the same herbal component using aqueous alcohol before the organic residue is recovered for that herbal component (per step (e)).

In a preferred embodiment, the process for making the herbal extract-based composition comprises the steps of:
(a) macerating hawthorn berries, macerating mulberry leaves, macerating bitter melon seeds and/or fruit, macerating green tea leaves and macerating Gynostemma pentaphyllum leaves into small pieces (e.g., millimeter size) while keeping each herbal component separate;
(b) placing the macerated hawthorn, macerated mulberry, macerated bitter melon, macerated green tea, and macerated Gynostemma pentaphyllum into separate containers;
(c) separately diluting each herbal component from step (b) in warm water, preferably at in a temperature range of between approximately 70° C. to 80° C., preferably in a ratio range of water to each herbal component of five to one;
(d) allowing each herbal component to soak in the warm water for at least 1 hour (preferably for 2-4 hours);
(e) collecting an extraction eluate from each soaking of the herbal components into separate receptacles;
(f) re-extracting the macerated hawthorn, macerated green tea, macerated bitter melon, macerated Gynostemma pentaphyllum, and macerated mulberry in a warm solution of water by repeating steps (c)-(d);
(g) collecting a re-extraction eluate from each re-extraction of each of the herbal components and pooling the re-extraction eluate with the extraction eluate of the respective herbal component;
   and in an extraction of a second batch of macerated hawthorn, macerated green tea, macerated Gynostemma pentaphyllum, macerated bitter melon, and macerated mulberry in an aqueous alcohol (e.g., 50% to 70% alcohol solution);
(h) placing the second batch of macerated hawthorn, macerated green tea, macerated Gynostemma pentaphyllum, macerated bitter melon, and macerated mulberry into separate containers;
(i) separately diluting each herbal component from step (h) in an aqueous alcohol solution (preferably, 70% ethanol; preferably at room temperature, e.g., a range of between approximately 25° C. to 40° C.; preferably in a ratio range of alcohol solution to each herbal component of five to one);
(j) allowing each herbal component to soak in the aqueous alcohol solution for at least 1 hour (preferably for 2-4 hours);
(k) collecting the aqueous alcohol solution extraction eluate from the soaking of each of the herbal components into separate receptacles;
(l) re-extracting the second batch of macerated hawthorn, macerated green tea, macerated bitter melon, macerated Gynostemma pentaphyllum, and macerated mulberry in an aqueous alcohol solution by repeating steps (i)-(j);
(m) collecting the aqueous alcohol solution re-extraction eluates from the soaking of each of the herbal components and pooling each aqueous alcohol solution supernatant re-extract with the aqueous alcohol solution extraction eluate of the respective herbal component;
(n) recovering the organic residue from the each pooled eluate (e.g., from step (g) and from step (m), separately (water extract vs. aqueous alcohol extract ) or pooled together (water extract pooled with aqueous alcohol extract)) of each herbal component by reducing the liquid portion of each of the respective pooled aqueous alcohol extraction eluate by drying (air drying, freeze drying, or a combination thereof) in forming a green tea extract powder, a Gynostemma pentaphyllum extract powder, a hawthorn extract powder, a mulberry extract powder, and a bitter melon extract powder; and
(o) combining the green tea extract powder, the Gynostemma pentaphyllum extract powder, the hawthorn extract powder, the mulberry extract powder, and the bitter melon extract powder in the desired proportions to form a herbal extract-based composition.

As an alternative to any of the processes embodied herein, for producing a bitter melon extract powder, the fruit of the bitter melon may be cut up into small pieces, which are then freeze-dried and powdered using standard methods known in the art. This technique involving freeze drying and powdering may also be used for producing a hawthorn extract powder from hawthorn berries, and a mulberry extract powder from the berries of mulberry.

In illustrating a preferred embodiment, and following the process for producing the herbal extract-based composition, the herbal extract-based composition may be formed by mixing comprises about 5 percent to about 20 percent by weight of Gynostemma pentaphyllum extract, about 20 percent to about 40 percent by weight of green tea extract, about 20 percent to about 40 percent by weight of hawthorn extract, about 20 percent to about 40 percent by weight of bitter melon extract; and optionally, about 0 percent to about 15 percent by weight of mulberry extract. Preferably, the composition comprises, as active components, green tea polyphenols, hawthorn polyphenols, saponins of Gynostemma pentaphyllum, and an extract of bitter melon; and may further comprise N-containing sugars of mulberry. Continuing this illustration of a preferred embodiment, using methods known in the art, the composition is formed into a tablet, wherein the combined weight of the extract powders comprises a weight in the range of about 250 mg to about 700 mg. In a preferred embodiment, such tablet form (including both active ingredients and inactive ingredients), and intrinsic to the herbal extract-based composition is typically found at least about 15% by weight of proanthocyanidins, at least about 5% by weight saponins, at least 15% by weight polyphenols, at least 5% by weight of mulberry extract, and at least 20% by weight of bitter melon components which have anti-diabetic effects (e.g., reduce hyperglycemia). The remaining weight percentage comprises inactive ingredients, including a base for facilitating the formation of the tablets. In such tablet form, a preferred dosage and regimen as a dietary supplement for an adult male or female to effect the health-promoting and/or therapeutic effects provided by the herbal extract-based composition of the present invention is 1 tablet, two to three times daily; and preferably shortly after meals.

From the foregoing, it will be obvious to those skilled in the art that various modifications in the above-described methods, and compositions can be made without departing from the spirit and scope of the invention. Accordingly, the invention may be embodied in other specific forms without departing from the spirit or essential characteristics thereof. Present embodiments and examples, therefore, are to be considered in all respects as illustrative and not restrictive, and all changes which come within the meaning and range of equivalency of the claims are therefore intended to be embraced therein.

What is claimed is:

1. A composition comprising about 5 percent to about 20 percent by weight of Gynostemma pentaphyllum extract, about 20 percent to about 40 percent by weight of Camellia sinensis (green tea) extract, about 20 percent to about 40 percent by weight of Crataegus pinnatifidia (hawthorn) extract, and about 20 percent to about 40 percent by weight of Momordica charantia (bitter melon) extract.

2. The composition according to claim 1, further comprising about 0 percent to about 15 percent by weight of mulberry (Morus species) extract.

3. The composition according to claim 1, wherein the composition comprises about 5 percent by weight of Gynostemma pentaphyllum extract, about 30 percent by weight of green tea extract, about 30 percent by weight of hawthorn extract, and about 30 percent by weight of bitter melon extract.

4. The composition according to claim 3, further comprising about 0 percent to about 10 percent by weight of mulberry extract.

5. A composition comprising about 5 percent to about 20 percent by weight of Gynostemma pentaphyllum saponins, about 20 percent to about 40 percent by weight of green tea polyphenols, about 20 percent to about 40 percent by weight of hawthorn polyphenols, and about 20 percent to about 40 percent by weight of bitter melon extract.

6. The composition according to claim 5, further comprising about 0 percent to about 10 percent by weight of N-containing sugars of mulberry.

7. The composition according to claim 5, wherein the composition comprises about 5 percent by weight of Gynostemma pentaphyllum saponins, about 30 percent by weight of green tea polyphenols, about 30 percent by weight of hawthorn polyphenols, and about 30 percent by weight of bitter melon extract.

8. The composition according to claim 7, further comprising about 0 percent to about 10 percent by weight of N-containing sugars of mulberry.

9. A composition comprising active ingredients, wherein the active ingredients are comprised of about 5 percent to about 20 percent by weight of Gynostemma pentaphyllum extract, about 20 percent to about 40 percent by weight of Camellia sinensis (green tea) extract, about 20 percent to about 40 percent by weight of Crataegus pinnatifidia (hawthorn) extract, and about 20 percent to about 40 percent by weight of Momordica charantia (bitter melon) extract.

10. The composition according to claim 9, wherein the active ingredients are further comprised of about 0 percent to about 15 percent by weight of mulberry (Morus species) extract.

11. The composition according to claim 9, wherein the active ingredients are comprised of about 5 percent by weight of Gynostemma pentaphyllum extract, about 30 percent by weight of green tea extract, about 30 percent by weight of hawthorn extract, and about 30 percent by weight of bitter melon extract.

12. The composition according to claim 11, wherein the active ingredients are further comprised of about 0 percent to about 10 percent by weight of mulberry extract.

13. The composition of any one of claims 9-12, wherein the active ingredients are contained in a form selected from the group consisting of a tea, a liquid-extract, a beverage, a gum, a lozenge, a powder, a tablet, or a lotion.

14. A composition comprising active ingredients, wherein the active Ingredients are comprised of about 5 percent to about 20 percent by weight of Gynostemma pentaphyllum saponins, about 20 percent to about 40 percent by weight of green tea polyphenols, about 20 percent to about 40 percent by weight of hawthorn polyphenols, and about 20 percent to about 40 percent by weight of bitter melon extract.

15. The composition according to claim 14, wherein the active ingredients are further comprised of about 0 percent to about 10 percent by weight of N-containing sugars of mulberry.

16. The composition according to claim 14, wherein the active ingredients are comprised of about 5 percent by weight of Gynostemma pentaphyllum saponins, about 30 percent by weight of green tea polyphenols, about 30 percent by weight of hawthorn polyphenols, and about 30 percent by weight of bitter melon extract.

17. The composition according to claim 16, wherein the active ingredients are further comprised of about 0 to about 10 pecent by weight of N-containing sugars of mulberry.

18. The composition of any one of claims 14-17, wherein the active ingredients are contained in a form selected from the group consisting of a tea, a liquid extract, a beverage, a gum, a lozenge, a powder, a tablet, or a lotion.

19. A process for making a composition comprising the steps of:
(a) separately macerating herbal components wherein a first component comprises hawthorn berries or leaves or a combination thereof, a second component comprises mulberry leaves or berries or a combination thereof, a third component comprises bitter melon leaves or seeds or fruit or a combination thereof, a fourth component comprises green tea leaves, and a fifth component comprises Gynostemma pentaphyllum leaves;
(b) separately extracting each macerated component from step (a) in warm water for at least 1 hour;
(c) collecting an extraction eluate from step (b) from each macerated component;
(d) re-extracting separately each macerated component from step (c) in warm water;
(e) collecting a re-extraction eluate from step (d) from each macerated component, and pooling together the re-extraction eluate with the extraction eluate from each macerated component;
(f) separately extracting macerated components comprised of macerated hawthorn, macerated green tea, macerated Gynostemma pentaphyllum, macerated bitter melon, and macerated mulberry, in an aqueous alcohol for at least 1 hour;
(g) collecting the aqueous alcohol solution extraction eluate from step (f) from each macerated component;
(h) re-extracting each macerated component from step (g) in an aqueous alcohol;
(i) collecting the aqueous alcohol solution re-extraction eluates from step (h) and pooling them with the aqueous alcohol solution extraction eluate from step (g) from the respective macerated component;
(j) drying the pooled extracts from steps (e) and (i) and recovering resultant organic residue in forming a green tea extract powder, a Gynostemma pentaphyllum extract powder, a hawthorn extract powder, a mulberry extract powder, and a bitter melon extract powder; and
(k) combining the green tea extract powder, the Gynostemma pentaphyllum extract powder, the hawthorn extract powder, the mulberry extract powder, and the bitter melon extract powder in the desired proportions to form a herbal extract-based composition.
